(19) United States
(12) Patent Application Publication
(10) Pub. No.: US 2025/0073380 A1
(43) Pub. Date: Mar. 6, 2025

(54) BREAST PUMP AND MILK COLLECTING ASSEMBLY

(71) Applicant: Shenzhen Lute Jiacheng Supply Chain Management Co., Ltd., Shenzhen (CN)

(72) Inventor: Jieqing Zheng, Shenzhen (CN)

(73) Assignee: Shenzhen Lute Jiacheng Supply Chain Management Co., Ltd., Shenzhen (CN)

(21) Appl. No.: 18/430,618

(22) Filed: Feb. 1, 2024

(30) Foreign Application Priority Data
Aug. 30, 2023 (CN) 202311112090.9
Aug. 30, 2023 (CN) 202322354456.5
Aug. 30, 2023 (CN) 202322355549.X
Sep. 4, 2023 (CN) 202322406037.1

(51) Int. Cl. A61M 1/06

(52) U.S. Cl. CPC A61M 1/064

(57) ABSTRACT

The disclosure provides a breast pump, including a milk collecting assembly and a bowl member; the milk collecting assembly comprises a hard shell and a soft rubber inner cover; the soft rubber inner cover is in contact with a breast of a user when in use; the soft rubber inner cover is disposed on one side of the hard shell towards the breast, and the hard shell is detachably connected to the bowl member. The soft rubber inner cover has deformation ability. Under negative pressure, the soft rubber inner cover can squeeze the breasts, and the collected milk can flow out of the soft rubber inner cover into the milk bowl.

### CROSS-REFERENCE TO RELATED APPLICATION

The present disclosure claims priority to Chinese Patent Application No. 202322406037.1, filed with the Chinese Patent Office on Sep. 4, 2023, titled “BREAST PUMP AND ASSEMBLY STRUCTURE OF BREAST MASK AND BOWL MEMBER,” to Chinese Patent Application No. 202311112090.9, filed with the Chinese Patent Office on Aug. 30, 2023, titled “BREAST PUMP,” to Chinese Patent Application No. 202322355549.X, filed with the Chinese Patent Office on Aug. 30, 2023, titled “BREAST PUMP, AND NEGATIVE PRESSURE CONDUCTIVE COMPONENT AND BREAST PUMP COMPONENT THEREOF,” and to Chinese Patent Application No. 202322354456.5, filed with the Chinese Patent Office on Aug. 30, 2023, titled “BREAST PUMP AND MILK COLLECTING ASSEMBLY THEREOF,” the entire contents of which are incorporated herein by reference.

### TECHNICAL FIELD

The present disclosure relates to the field of maternal and infant products, and particularly to a milk collecting assembly and a breast pump comprising the same.

### BACKGROUND

Breast pump is a device for lying-in woman to use after childbirth. When conditions are not allowed to feed the baby with fresh breastmilk, lactating women can use a breast pump to suck breastmilk from the breasts and then store it for later use, which can avoid the waste of breastmilk and also facilitate the work and life of lactating women, and thus the breast pump is widely popular by the market.

Typically, an electric breast pump includes a suction cup, a milk storage container, and a main unit. The main unit operates to generate negative pressure to suck breast milk from the breasts and store the milk in the milk storage container connected to the suction cup.

The suction cup is in direct contact with the human body. To increase comfort, the suction cup is generally one-step molded with soft rubber material, while the bowl member is generally made of hard material. The outer edge of the suction cup is attached to the outer edge of the bowl member. In the process of connecting or disconnecting the suction cup and the bowl member, since the suction cup is made of soft rubber material, it is necessary to press or unfasten the entire outer edge of the suction cup that is in contact with the bowl member to complete the connection or disconnection of the suction cup and the bowl member. The connection or disconnection process is inefficient, and after installation, the connection therebetween is not strong enough.

### SUMMARY

In view of the shortcomings of the prior art, the disclosure provides a milk collecting assembly and a breast pump comprising the same, aiming to solve the problem of the complicated assembly and disassembly process of the suction cup and the milk collecting assembly in the art. To achieve the above objectives, the following technical solution are adopted:

A breast pump comprises: a milk collecting assembly and a bowl member; the milk collecting assembly comprises a hard shell and a soft rubber inner cover; the soft rubber inner cover is in contact with a breast of a user when in use; the soft rubber inner cover is disposed on one side of the hard shell towards the breast, and the hard shell is detachably connected to the bowl member.

The disclosure further provides another breast pump, comprising: a suction cup, a bowl member, and a main unit assembly; the suction cup comprises a hard shell and a soft rubber inner cover; the soft rubber inner cover is in contact with a breast of a user when in use; the soft rubber inner cover is disposed on one side of the hard shell towards the breast; the hard shell is detachably connected to the bowl member; and the main unit assembly is disposed on one side of the bowl member back against the hard shell.

Another objective of the disclosure is to provide a milk collecting assembly, comprising a flange and a milk suction tube which are integrally formed using hard materials; the flange comprises a breast accommodating portion, and the milk suction tube comprises a milk channel connected to the breast accommodating portion.

The milk collecting assembly of the disclosure comprise a hard shell and a soft rubber inner cover, and the hard shell is detachably connected to the milk bowl. The soft rubber inner cover is in contact with a breast of a user when in use, with deformation ability. Under negative pressure, the soft rubber inner cover can squeeze the breasts, and the collected milk can flow out of the soft rubber inner cover into the milk bowl.

### BRIEF DESCRIPTION OF THE DRAWINGS

FIG. 1 is a schematic diagram of a breast pump according to one embodiment of the disclosure;

FIG. 2 is a first exploded view of a breast pump according to one embodiment of the disclosure;

FIG. 3 is a first exploded view of a breast pump according to one embodiment of the disclosure;

FIG. 4 is a first exploded view of a breast pump according to one embodiment of the disclosure;

FIG. 5 is a longitudinal sectional view of a combination of a milk collecting assembly and a bowl member according to one embodiment of the disclosure;

FIG. 6 is an enlarged view of part A in FIG. 5;

FIG. 7 is a schematic diagram of a hard shell according to one embodiment of the disclosure;

FIG. 8 is a schematic diagram of a bowl member according to one embodiment of the disclosure;

FIG. 9 is a combination of a milk collecting assembly and a bowl member according to one embodiment of the disclosure;

FIG. 10 is a longitudinal section of a diaphragm according to one embodiment of the disclosure;

FIG. 11 is a schematic diagram of a main unit assembly according to one embodiment of the disclosure;

FIG. 12 is a longitudinal sectional view of a breast pump according to one embodiment of the disclosure; and

FIG. 13 is an enlarged view of part B in FIG. 12.

The objectives, functional features and advantages of the disclosure will be further described in conjunction with the embodiments and with reference to the accompanying drawings.

### DETAILED DESCRIPTION

In the disclosure, the terms “dispose”, “provided with” and “connect” are to be understood in a broad sense. For example, it may be a fixed connection, a detachable connection, or a monolithic construction; it may be a mechanical connection or an electrical connection; it may be a direct connection or an indirect connection through an intermediate; or it may be an internal connection between two devices, elements, or components. To one of ordinary skill in the art, the specific meaning of the above terms in the disclosure may be determined according to specific circumstances.

The terms “center”, “longitudinal”, “transverse”, “length”, “width”, “thickness”, “on”, “below”, “front”, “rear”, “left”, “right”, “vertical”, “horizontal”, “top”, “bottom”, “inner”, “outer”, “clockwise”, “counterclockwise”, “axial”, “radial”, “circumferential” and the like indicate orientations or positional relationships based on those shown in the accompanying drawings, and are intended only to facilitate the description of the disclosure and to simplify the description, and are not intended to indicate or imply that the device or element referred to must have a particular orientation, be constructed and operated in a particular orientation, and therefore are not to be construed as a limitation of the disclosure.

Furthermore, the terms “first” and “second” are used only for descriptive purposes and are not to be construed as indicating or implying relative importance or implicitly specifying the number of technical features indicated. Thus, an element defined with the terms “first”, “second” may expressly or impliedly include at least one such element. In the description of the disclosure, “plurality” means at least two, e.g., two, three, etc., unless otherwise specifically stated.

Moreover, some of the above terms may be used to indicate other meanings in addition to an orientation or positional relationship, for example, the term “on” may also be used in some cases to indicate a certain kind of dependency or connection relationship. To one of ordinary skill in the art, the specific meaning of these terms in the disclosure may be understood according to the specific circumstances.

To make the objectives, technical solutions and advantages of the disclosure clearer and more understandable, the disclosure is hereinafter described in further detail in conjunction with the accompanying drawings and embodiments. It should be understood that the specific embodiments described herein are only for explaining the disclosure and are not intended to limit the disclosure.

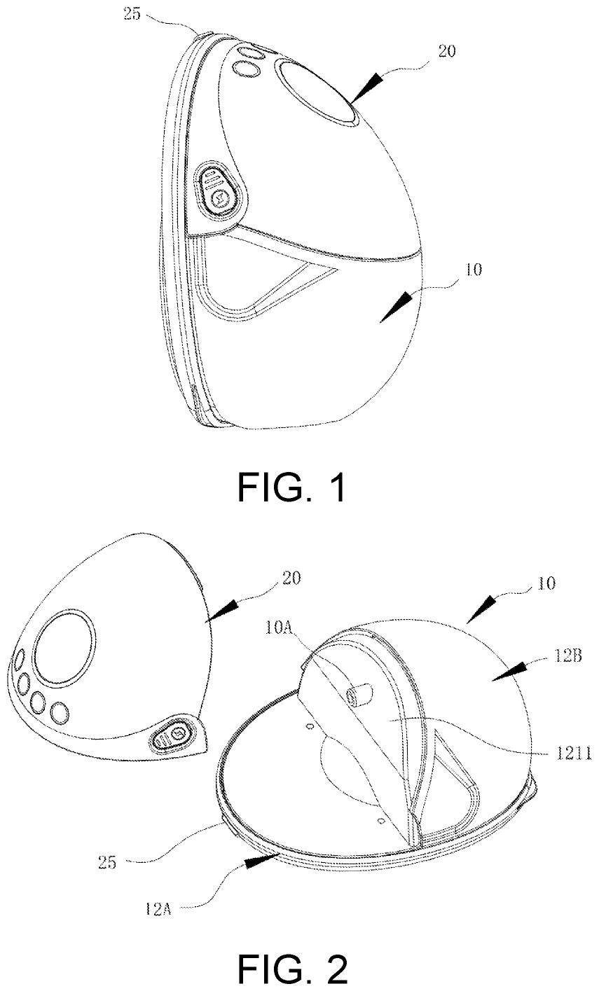

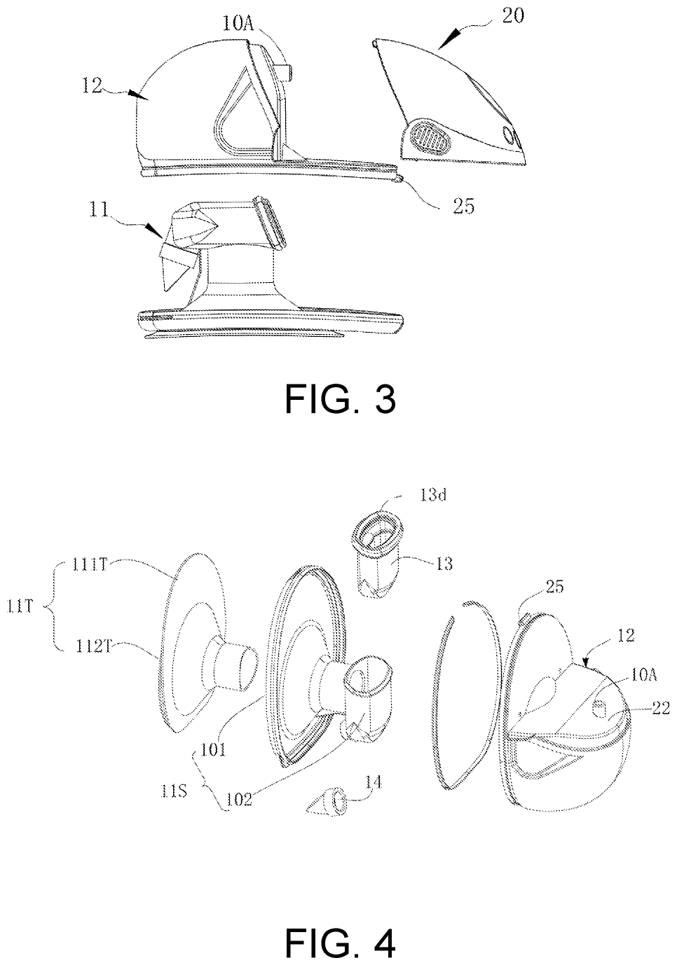

As shown in FIGS. 1-4, the disclosure provides a breast pump comprising a milk collecting assembly 11 and a bowl member 12. The milk collecting assembly 11 comprises a hard shell 11S and a soft rubber inner cover 11T; the soft rubber inner cover 11T is in contact with a breast of a user when in use; the soft rubber inner cover 11T is disposed on one side of the hard shell 11S towards the breast, and the hard shell 11S is detachably connected to the bowl member 12.

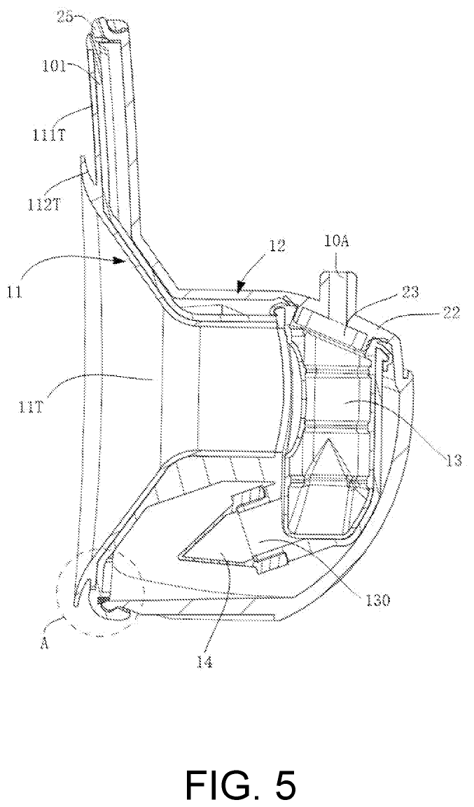

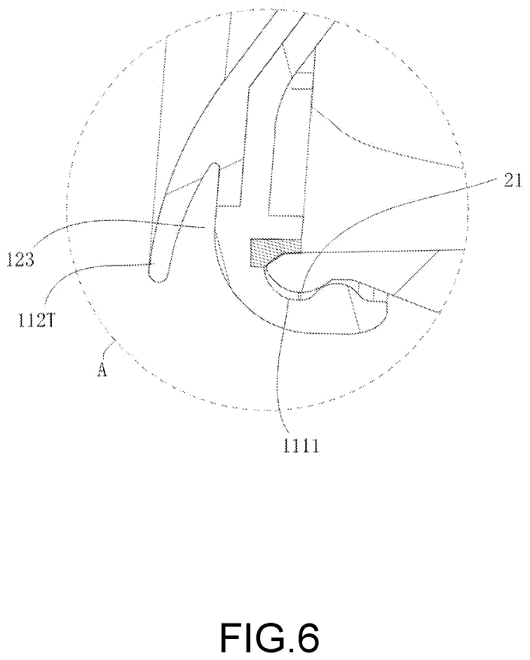

Exemplarily, as shown in FIGS. 5 and 6, the hard shell 11S comprises a first outer edge, and the bowl member 12 comprises a second outer edge; and the first outer edge is interlocked with the second outer edge.

In practice, the soft rubber inner cover 11T may be made of silicone material. Since the hard shell 11S features high rigidity, high torque, and good force transmission effect compared to the soft rubber material, when combining the hard shell 11S with the bowl member 12, only need to press a part of the outer edge of the hard shell 11S, the outer edge of the hard shell 11S is attached to the outer edge of the bowl member 12 in the snap-fit connection; when separating the hard shell 11S and bowl member 12, only need to turn part of the outer edge of the hard shell 11S outward thus completely detaching the hard shell 11S from the bowl member 12. Understandably, since the hard shell 11S is a combination of hard and soft materials, it is deformable, which, on one hand, facilitates the milking operation, and on the other hand, is easy for users to install and uninstall, so it is convenient for users to clean the hard shell 11S and the bowl member 12 in their daily lives.

In certain embodiment, the soft rubber inner cover 11T comprises a cover body 111T and a rim 112T; the cover body 111T is flared for directly covering a breast; the soft rubber inner cover 11T is tightly affixed to one side of the hard shell 11S facing the breast; the rim 112T is disposed on one side of the cover body 111T facing the breast. There is a deformation gap 123 between the rim 112T and the cover body 111T. Owing to the presence of the deformation gap 123, the rim 112T is provided with sufficient deformable space, so that the rim 112T can match the breast sufficiently in shape, and be adsorbed on the breast closely, avoiding air leakage, and effectively ensuring the strength of negative pressure.

In certain embodiment, the hard shell 11S comprises a flange 101 and a three-way pipe 102; the outer edge of the flange 101 is interlocked with the outer edge of the three-way pipe 102. As shown in FIG. 6, the inner side of the outer edge of the flange 101 comprises a first groove 1111, and the outer side of the outer edge of the bowl member 12 comprises a first protrusion 21. The first groove 1111 is engaged with the first protrusion 21 through snap-fit connection. Since the first groove 1111 has a wrapping effect on the outer edge of the bowl member 12, no raised structure extends out of the outer edge of the bowl member 12 to contact the human body, thus improving the comfort of the user when using the breast pump. Understandably, the first groove 1111 is a continuous annular groove or a plurality of small slots spaced circumferentially along the outer edge of the flange 101. Similarly, the first protrusion 21 may be a continuous annular structure or a plurality of small protrusions spaced circumferentially along the outer edge of the bowl member 12.

Understandably, to realize the interlocking of the outer edge of the flange 101 and the outer edge of the bowl member 12, in other embodiments, it is also possible to provide the first protrusion 21 on the inner side of the outer edge of the flange 101, and the first groove 1111 on the outer side of the outer edge of the bowl member 12, so that the first protrusion 21 is clamped in the first groove 1111.

In actual use, to ensure that the hard shell 11S has a certain degree of structural strength, while avoiding excessive mass, the hard shell 11S can be made of rigid plastic, optionally, can also be used in alloy or stainless steel and other lightweight and non-rusting metal materials.

In certain embodiments, the flange 101 is integrated with the three-way pipe 102 and is detachably connected to the bowl member 12; and the three-way pipe 102 is connected to the flange 101, the bowl member 12, and a negative pressure source.

For example, the flange 101 is integrated with the three-way pipe 102 through injection molding using rigid plastic, which, compared with an assembled structure by the flange 101 and the three-way pipe 102, not only improves the production efficiency of the parts of the breast pump, but also reduces the number of parts of the breast pump, and further facilitates the user to assemble and disassemble the breast pump.

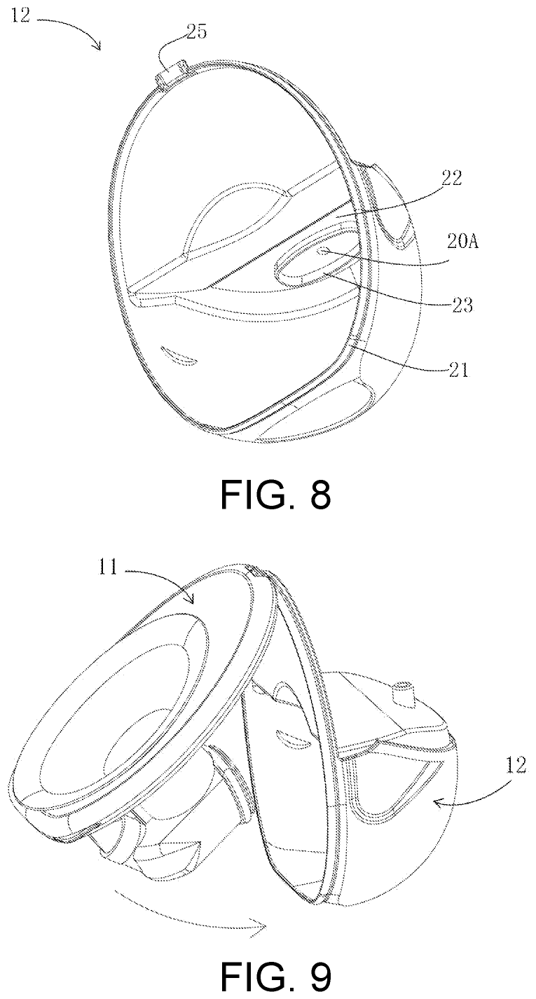

Referring to FIGS. 5 and 7-9, the outer edge of the bowl member 12 is projected in an outward direction to form a milk release mouth 25; the milk release mouth 25 communicates with the inside of the bowl member 12, and the milk collected inside the bowl member 12 can flow out through the milk release mouth 25; as shown in FIG. 1, the milk release mouth 25 of this embodiment is disposed on the top of the bowl member 12. In this embodiment, when the breast pump is used for milk collection, the milk release mouth 25 is facing upward, and the milk can enter the milk storage space through the flange 101 and the three-way pipe 102; when it is necessary to pour out the milk, there is no need to separate the hard shell 11S from the bowl member 12, and only by tilting the breast pump, the milk can naturally flow out of the milk storage space through the milk release mouth 25.

In certain embodiments, a through hole 1112 is disposed at the top of the outer edge of the flange 101. When combining the hard shell 11S and the bowl member 12, the milk release mouth 25 is embedded into the through hole 1112 to limit the hard shell 11S. Optionally, the hard shell 11S is rotatable with the milk release mouth 25 as a fulcrum. After rotating a preset angle, the outer edge of the flange 101 can be interlocked with the outer edge of the bowl member 12. Understandably, the through hole 1112 can be an opening or a gap that can limit the milk release mouth 25. For example, in another embodiment, the through hole 1112 is a gap. When combining the hard shell 11S and bowl member 12, the milk release mouth 25 is embedded in the gap first, and then, without rotating the hard shell 11S, the outer edge of the flange 101 is directly engaged with the outer edge of the bowl member 12.

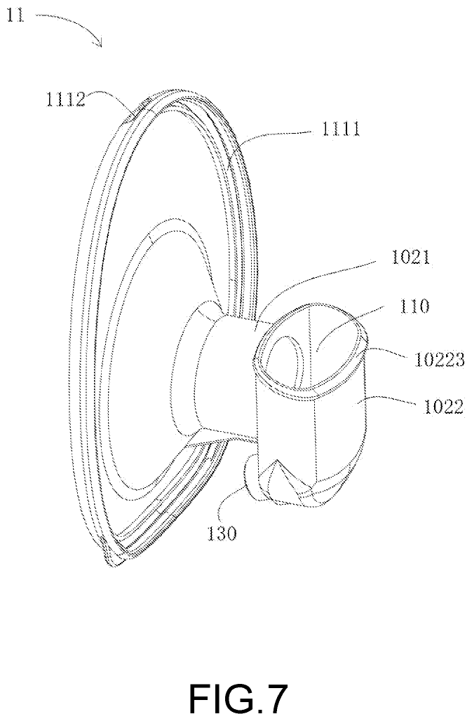

As shown in FIG. 7, the three-way pipe 102 comprises a milk suction tube 1021 and a bracket 1022. One end of the milk suction tube 1021 is fixedly connected to one side of the flange 101 away from the breast, and the milk suction tube 1021 communicates with the inner space of the flange 101. The bracket 1022 comprises a negative pressure port 110 and a milk outlet 130 communicating with the negative pressure port 110; the negative pressure port 110 functions for the entrance or exiting of the air, and the other end of the milk suction tube 1021 is connected to the bracket 1022; the milk suction tube 1021 communicates with the negative pressure port 110 of the bracket 1022, and the milk outlet 130 communicates with the inner space of the bowl member 12. Since the soft rubber inner cover 11T is deformable, it can squeeze the breast under the negative pressure; the inner space of the soft rubber inner cover 11T communicates with the milk suction tube 1021 of the three-way pipe 102; the collected milk can flow out from the soft rubber inner cover 11T to the milk suction tube 1021, and flow into the bowl member 12 through the milk outlet 130 of the three-way pipe 102.

In certain embodiments, the hard shell 11S comprises a bracket 1022 with a negative pressure chamber; the negative pressure acting on the negative pressure chamber may be transmitted to the space between the soft rubber inner cover 11T and the breast to supply negative pressure for the space to suck the milk.

Referring to FIGS. 5 and 7, in certain embodiments, the bracket 1022 comprises a top end with an inclined opening portion 10223 surrounding the negative pressure chamber; the top wall of the bowl member 12 comprises an inclined portion 22 corresponding to the inclined opening portion 10223, and the inclined opening portion 10223 abuts against the inclined portion 22. Exemplarily, the side of the inclined opening portion 10223 away from the flange 101 slopes downwardly (as in the downward direction in FIGS. 5 and 7) relative to the side close to the flange 101. The inner surface of the top wall of the bowl member 12 comprises the inclined portion 22 corresponding to the inclined opening portion 10223; and the inclined opening portion 10223 is in close contact with the inclined portion 22.

As shown in FIGS. 4, 5, 7, and 10, the milk collecting assembly 11 further comprises a diaphragm 13; the three-way pipe 102 comprises a milk suction tube 1021 and a bracket 1022; the milk suction tube 1021 comprises a milk channel connected to the flange 101; the bracket 1022 comprises a negative pressure chamber connected to the milk channel, and the diaphragm 12 is disposed in the negative pressure chamber.

Exemplarily, the bracket 1022 cooperates with the top wall of the bowl member 12 to seal the diaphragm 13 in the negative pressure chamber. In one embodiment, the diaphragm 13 is in the form of a cylinder with one end closed, and the open end of the diaphragm 13 comprises a convex edge 13d protruding outwardly; the convex edge 13d encloses one end of the hard shell 11S, and the hard shell 11S presses the convex edge 13d against the inner surface of the top wall of the bowl member 12 so that the open end of the diaphragm 13 is connected to a negative pressure source through a negative pressure connector 10A on the bowl member 12.

In addition, the bracket 1022 further comprises a milk outlet 130 connecting the milk channel and the bowl member 12; during a milk collection process, the milk outlet 130 is disposed on the lower part of the negative pressure chamber. Furthermore, the milk suction tube 1021 is connected to the side wall of the bracket 1022. Specifically, the bracket 1022 is connected to one end of the milk suction tube 1021 away from the flange 101, and the milk channel communicates with the negative pressure chamber at one end of the milk channel.

The diaphragm 13 functions to seal and isolate the fluid medium between the negative pressure port 110 and the milk outlet 130, as well as to seal and isolate the fluid medium between the negative pressure port 110 and the milk channel. The diaphragm 13 also functions to transfer the negative pressure at the negative pressure port 110 to the milk channel.

In this way, the hard shell 11S is provided with the milk channel, the negative pressure port 110, and the milk outlet 130; the milk channel is used to hold the breast and receive milk; the negative pressure port 110 is connected to a negative pressure source, and the milk outlet 130 is connected to the bowl member 12; the cyclic negative pressure exerted by the negative pressure source on the diaphragm 13 causes the diaphragm 13 to produce cyclic deformations and generate a suction force for milk, and thus the milk of the human breast is drawn by the suction force to the milk channel, flows through the milk channel and then out from the milk outlet 130, and is collected in the bowl member 12 for storage.

As shown in FIGS. 5-7, in one embodiment, an annular groove 23 is provided on the inner side of the inclined portion 22, and the sidewall of the annular groove 23 may be perpendicular to the inner surface of the inclined portion 22. The inclined opening portion 10223 is rested on the outer edge of the annular groove 23, and the annular groove 23 is provided with a negative pressure connector 10A for supplying or discharging air into or out of the bracket 1022. For example, the inclined opening portion 10223 of the bracket 1022 and the inclined portion 22 of the bowl member 12 are tilted and corresponding to each other, the spatial interference between the top of the bracket 1022 and the annular groove 23 is avoided during rotational installation of the hard shell 11S. In addition, through the above corresponding structure design, the contact between the inclined opening portion 10223 and the inclined portion 22 is closer, which strengthens the tightness of the air passage and avoids air leakage that may lead to insufficient negative pressure.

Specifically, during installing the diaphragm 13, the opening part at the top of the diaphragm 13 sleeves the outer edge of the annular groove 23 and abuts against the inner side of the inclined portion 22 of the bowl member 12 to achieve the sealed contact with the inclined portion 22, and the diaphragm 13 is in communication with the negative pressure connector 10A. The diaphragm 13 expands or contracts by indrawing or discharging air, thereby injecting or drawing out air into or from the inside of the soft rubber inner cover 11T through the negative pressure port 110. When the air inside the soft rubber inner cover 11T is drawn out, a negative pressure is formed inside the soft rubber inner cover 11T, and an elastic deformation occurs to squeeze the breast.

As shown in FIG. 17, the disclosure also provides a milk collecting assembly 11, comprising a flange 101 and a milk suction tube 1021 which are integrally formed using hard materials; the flange 101 comprises a breast accommodating portion, and the milk suction tube 1021 comprises a milk channel connected to the breast accommodating portion.

In certain embodiment, the hard shell 11S further comprises a bracket 1022 integrally formed with the flange 101 and the milk suction tube 1021; the bracket 1022 comprises a negative pressure chamber and a milk outlet 130, and both the negative pressure chamber and the milk outlet 120 communicate with the milk channel. Specifically, the milk suction tube 1021 can also be disposed to connect to the side wall of the negative pressure chamber, so that the negative pressure chamber is located at one end of the milk suction tube 1021 away from the flange 101.

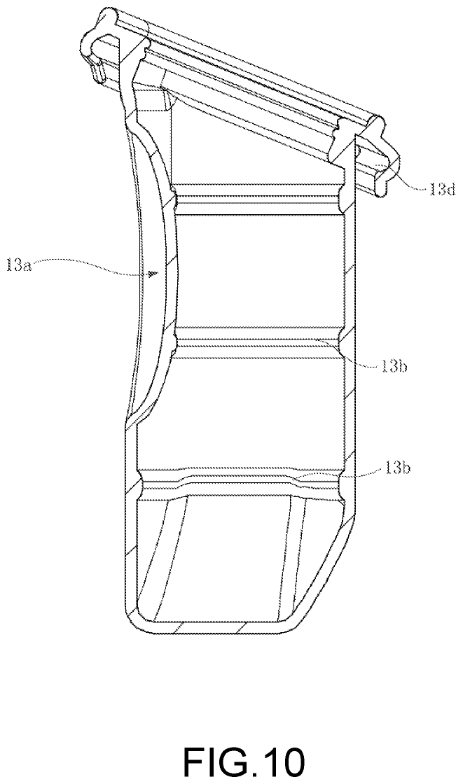

As shown in FIGS. 5 and 10, in one embodiment, one side of the diaphragm 13 facing the milk channel comprises a recess 13a, and a hollow space of the recess communicates with the milk channel.

The recess 13a can increase the deformation of the diaphragm 13 under negative pressure, thereby increasing the suction force generated by the deformation of the diaphragm 13. Compared with the diaphragm 13 without the recess 13a, the diaphragm 13 in this embodiment can generate greater suction force under a smaller negative pressure, avoiding the situation where the suction force is too small to suck out milk. In addition, the diaphragm 13 of the disclosure is disposed on the side near the milk channel, so that the diaphragm 13 is directly facing the milk channel, reducing the loss of force of the diaphragm 13 during the transmission of the negative pressure.

Further, the diaphragm 13 is provided with reinforcing bars 13b, which can strengthen the resilience of the diaphragm 13. In one embodiment, the number of reinforcing bars 13b is plural, and the plurality of reinforcing bars 13b are disposed at intervals.

As shown in FIG. 5, the milk outlet 130 of the hard shell 11S is provided with a one-way valve 14, which allows the milk in the milk channel to flow out of the milk outlet 130 below the bracket 1022 in a one-way direction into the bowl member 12, thus avoiding the backflow of the milk in the bowl member 12.

As shown in FIGS. 1-3, in one embodiment, the breast pump further comprises a main unit assembly 20; the main unit assembly 20 is disposed on one side of the bowl member 12 back against the milk collecting assembly 11, and the bowl member 12 separates the main unit assembly 20 from the milk collecting assembly 11. A negative pressure source may be disposed in the main unit assembly 20.

The milk collecting assembly 11 cooperates with the bowl member 12 to form a wearing assembly 10; the wearing assembly 10 is used to be worn on the breast part of the human body, and a milk storage space is formed therein. The main unit assembly 20 is used to provide a suction force for the wearing assembly 10 to suck up the milk from the breast and store it. The side of the wearing assembly 10 close to the human body is the wearing surface, and the wearing surface comprises a milk collection port communicating with the milk storage space. The main unit assembly 20 is connected to the milk storage space to provide the milk storage space with a negative pressure that generates suction force, and when the main unit assembly 20 is in operation, the milk can be collected from the milk collection port to the milk storage space.

Specifically, the wearing assembly 10 comprises a supporting portion 12A and a raised portion 12B; the supporting portion 12A comprises a wearing surface, and the raised portion 12B protrudes from a portion of the supporting portion 12A back against the wearing surface; the main unit assembly 20 is disposed on one side of the supporting portion 12A back against the milk collecting assembly 11 and is detachably connected to the raised portion 12B.

In this way, the raised portion 12B is formed at one end of the supporting portion 12A, and the other end may be used to receive the main unit assembly 20. When the main unit assembly 20 is mounted on the supporting portion 12A, the main unit assembly 20 rests against the raised portion 12B, such that the outer peripheral surface of the joint of the main unit assembly and the raised portion is smooth, thus forming a structurally stable assembly. For example, the main unit assembly 20 is connected to the raised portion 12B through a smooth transition to form a flat or curved surface which is smooth or substantially smooth. As shown in FIG. 1, the outer peripheral surface of the main unit assembly 20 and the outer peripheral surface of the raised portion 12B are curved surfaces, the supporting portion 12A is oval, and the assembled breast pump is a semi-ellipsoid or hemisphere.

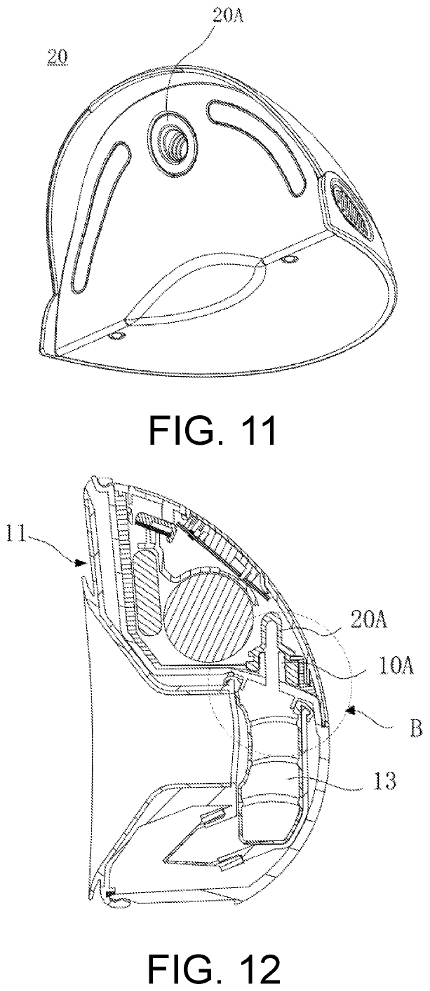

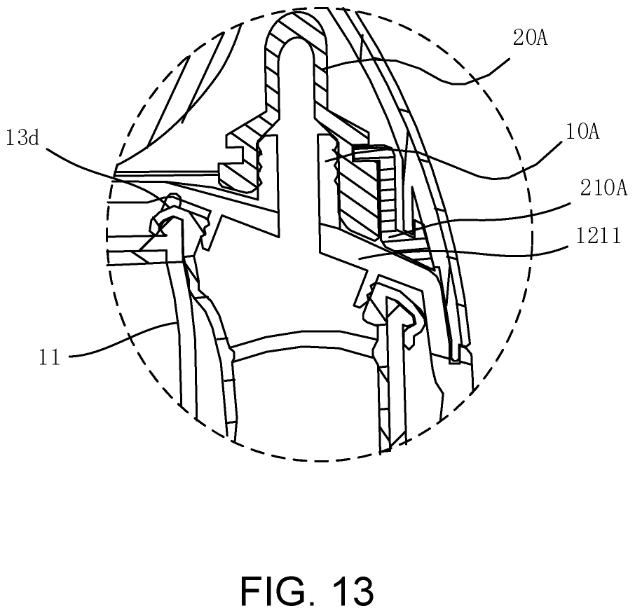

As shown in FIGS. 11-13, the main unit assembly 20 is detachably connected to the wearing assembly 10. Referring to FIGS. 1-3 and FIGS. 11-13, the main unit assembly 20 is disposed above the milk bowl 12, so that the center of gravity of the breast pump is above the milk bowl 12. In the use of the breast pump, the wearing assembly 10 is worn on the breast of the human body, and the main unit assembly 20 has a certain weight due to the presence of circuit boards, negative pressure sources and other heavy structural parts, which can enable the breast pump to be better attached to the breast, thus avoiding the tilting of the top of the breast pump due to less weight and improving the wearing comfort.

Specifically, the main unit assembly 20 comprises a negative pressure source 21B for generating negative pressure and a negative pressure socket 20A connected to the negative pressure source 21B. The negative pressure socket 20A is exposed out of the surface of the main unit assembly 20 facing the raised portion 12B and is located on the bottom of the main unit assembly 20. Correspondingly, the top wall of the milk bowl 12 is equipped with a negative pressure connector 10A; the negative pressure connector 10A is connected to the raised portion 12B and faces the main component 20. After the main unit assembly is combined with the milk bowl 12, the negative pressure connector 10A is inserted into the negative pressure socket 20A to connect to the negative pressure source 21B.

In this way, the negative pressure connector 10A is coupled and connected with the negative pressure socket 20A, so that on the one hand, the negative pressure can be transmitted, and on the other hand, a detachable connection between the main unit assembly 20 and the wearing assembly 10 can also be realized. In one embodiment, the negative pressure connector 10A and the negative pressure socket 20A have an interference fit, and the two elements have a good sealing effect at the joint. With the interference fit of the negative pressure connector 10A and the negative pressure socket 20A, the main unit assembly 20 and the wearing assembly 10 can be quickly combined or separated as needed. Exemplarily, the negative pressure socket 20A is made of soft rubber, and when a column-shaped negative pressure connector 10A is inserted into the negative pressure socket 20A, the negative pressure socket 20A is squeezed and stays in a tightened state, and if it is necessary to pull out the negative pressure connector 10A from the negative pressure socket 20A, a certain external force is required to be applied on the negative pressure connector 10A.

For example, one of the inner surface of the negative pressure socket 20A and the outer surfaces of the negative pressure connector 10A is formed with a plurality of annular convex ribs; when the negative pressure connector 10A is inserted into the negative pressure socket 20A, the annular convex ribs are resiliently located on the negative pressure connector 10A, which provides a certain tightening force to avoid the main unit assembly 20 from separating from the wearing assembly 10 easily. It is to be understood that in other embodiments, the outer surface of the negative pressure connector 10A may be provided with a soft rubber material, or a sealing member that can undergo deformation is provided between the negative pressure connector 10A and the negative pressure socket 20A, so as to enhance the reliability and sealing of the combination of the negative pressure connector 10A and the negative pressure socket 20A.

Understandably, the detachable connection between the main unit assembly 20 and the wearing assembly 10 is not limited to the above description. For example, the main unit assembly 20 and the wearing assembly 10 can be connected together by plugging, that is, there is a plug-in part on one of the main unit assembly 20 and the wearing assembly 10, and a socket on the other. The plug-in part is inserted into the socket to achieve the connection between the main unit assembly 20 and the wearing assembly 10. Alternatively, the main unit assembly 20 and the wearing assembly 10 can also be connected together through a snap-fit connection method. For example, one of the main unit assembly 20 and the wearing assembly 10 is equipped with a buckle, and the other is equipped with a hole. The buckle is held in the hole to achieve the connection between the main unit assembly 20 and the wearing assembly 10. Alternatively, the main unit assembly 20 and the wearing assembly 10 can also be connected together through connectors such as screws.

In one embodiment, the main unit assembly 20 comprises a tilted surface 210A tilted at an angle toward the raised portion 12B, and the tilted surface 210A fits with the inclined portion 1211. When the main unit assembly 20 is combined with the wearing assembly 10, the main unit assembly 20 is approaching toward the raised portion 12B, and the inclined portion 1211 of the raised portion 12B fits with the tilted surface 210A of the main unit assembly 20, guiding the main unit assembly 20 to slide along the inclined portion 1211 until the negative pressure connector 10A is inserted in the negative pressure socket 20A to reach a junction state.

Alternatively, the negative pressure connector 10A is disposed on the inclined portion 1211, the negative pressure socket 20A is disposed on the tilted surface 210A, and the extension direction of the negative pressure socket 20A is substantially the same as the extension direction of the supporting portion 12A, so that after the tilted surface 210A slides to correspond to the inclined portion 1211, the negative pressure connector 10A may be naturally inserted into the negative pressure socket 20A.

The disclosure further provides another breast pump, comprising: a suction cup 1, a bowl member 12, and a main unit assembly 20; wherein, the suction cup 1 comprises a hard shell 11S and a soft rubber inner cover 11T; the soft rubber inner cover 11T is in contact with a breast of a user when in use; the soft rubber inner cover 11T is disposed on one side of the hard shell 11S towards the breast; the hard shell 11S is detachably connected to the bowl member 12; and the main unit assembly 20 is disposed on one side of the bowl member 12 back against the hard shell 11S.

The above description is only the specific implementation method of this disclosure. It should be noted that for ordinary technical personnel in this field, several improvements and modifications can be made without departing from the principles of the disclosure. These improvements and modifications should also be considered as the scope of protection of this disclosure.

What is claimed is:

1. A breast pump comprising: a milk collecting assembly and a bowl member; wherein, the milk collecting assembly comprises a hard shell and a soft rubber inner cover; the soft rubber inner cover is in contact with a breast of a user when in use; the soft rubber inner cover is disposed on one side of the hard shell towards the breast, and the hard shell is detachably connected to the bowl member.

2. The breast pump of claim 1, wherein the hard shell comprises a first outer edge, and the bowl member comprises a second outer edge; and the first outer edge is interlocked with the second outer edge.

3. The breast pump of claim 1, wherein the hard shell comprises a bracket with a negative pressure chamber; the bracket comprises a top end with an inclined opening portion surrounding the negative pressure chamber; an inner surface of a top wall of the bowl member comprises an inclined portion corresponding to the inclined opening portion; and the inclined opening portion is in close contact with the inclined portion.

4. The breast pump of claim 1, wherein the hard shell comprises a flange and a three-way pipe; the flange is integrated with the three-way pipe and is detachably connected to the bowl member; and the three-way pipe is connected to the flange, the bowl member, and a negative pressure source.

5. The breast pump of claim 4, wherein the milk collecting assembly further comprises a diaphragm; the three-way pipe comprises a milk suction tube and a bracket; the milk suction tube comprises a milk channel connected to the flange; the bracket comprises a negative pressure chamber connected to the milk channel, and the diaphragm is disposed in the negative pressure chamber.

6. The breast pump of claim 5, wherein the bracket further comprises a milk outlet connecting the milk channel and the bowl member; during a milk collection process, the milk outlet is disposed on a lower part of the negative pressure chamber.

7. The breast pump of claim 5, wherein one end of the milk suction tube is connected to a side wall of the bracket.

8. The breast pump of claim 5, wherein the milk channel comprises communicates with the negative pressure chamber at one end of the milk channel.

9. The breast pump of claim 5, wherein the bracket is connected to one end of the milk suction tube away from the flange.

10. The breast pump of claim 5, wherein the bracket is matched with a top wall of the bowl member to seal the diaphragm in the negative pressure chamber.

11. The breast pump of claim 1, further comprising a main unit assembly; wherein the main unit assembly is disposed on one side of the bowl member back against the milk collecting assembly, and the bowl member separates the main unit assembly from the milk collecting assembly.

12. The breast pump of claim 11, wherein the milk collecting assembly cooperates with the bowl member to form a wearing assembly; the wearing assembly comprises a supporting portion and a raised portion; the supporting portion comprises a wearing surface, and the raised portion protrudes from a portion of the supporting portion back against the wearing surface; and the motor assembly is disposed on one side of the supporting portion back against the milk collecting assembly and is detachably connected to the raised portion.

13. The breast pump of claim 12, wherein when the motor assembly is disposed on the supporting portion, the main unit assembly abuts against the raised portion; and an outer surface of a joint of the main unit assembly and the raised portion is smooth.

14. A breast pump, characterized by comprising: a suction cup, a bowl member, and a main unit assembly; wherein, the suction cup comprises a hard shell and a soft rubber inner cover; the soft rubber inner cover is in contact with a breast of a user when in use; the soft rubber inner cover is disposed on one side of the hard shell towards the breast; the hard shell is detachably connected to the bowl member; and the motor assembly is disposed on one side of the bowl member back against the hard shell.

15. A milk collecting assembly, comprising a flange and a milk suction tube which are integrally formed, wherein the flange comprises a breast accommodating portion, and the milk suction tube comprises a milk channel connected to the breast accommodating portion.

16. The milk collecting assembly of claim 15, further comprising a bracket integrally formed with the flange and the milk suction tube; the bracket comprises a negative pressure chamber and a milk outlet, and both the negative pressure chamber and the milk outlet communicate with the milk channel.

17. The milk collecting assembly of claim 16, wherein one end of the milk suction tube is connected to a side wall of the bracket.

18. The milk collecting assembly of claim 16, wherein the milk channel communicates with the negative pressure chamber at one end of the milk channel.

19. The milk collecting assembly of claim 16, wherein each of the bracket, the flange and the milk suction tube comprises hard material.

20. The milk collecting assembly of claim 15, further comprising a soft rubber inner cover attached to the flange.
